(19) United States
(12) Patent Application Publication
(10) Pub. No.: US 2008/0085547 A1
(43) Pub. Date: Apr. 10, 2008

(54) Biofilter media and systems and methods of using same to remove odour causing compounds from waste gas streams

(76) Inventors: Brian P. Herner, Georgetown (CA); Zarook M. Shareefdeen, Guelph (CA)

Correspondence Address: FASKEN MARTINEAU DUMOULIN LLP, 4200 TORONTO DOMINION BANK TOWER, BOX 20 TORONTO-DOMINION CENTRE, TORONTO, ON, M5K 1N6, US

(21) Appl. No.: 11/542,107

(22) Filed: Oct. 4, 2006

(51) Int. Cl. A61L 9/01

(52) U.S. Cl. 435/266

(57) ABSTRACT

The present invention relates to biofilter systems and the biofilter media employed in such systems, as well as, methods of using same to remove odour causing compounds from waste gas streams. The biofilter media has a plurality of expanded glass granules. Each expanded glass granule has a coating thereon. The coating includes a bonding agent, an adsorptive agent, microorganisms and nutrients. When used in a biofilter system, the biofilter media is highly efficient at removing from waste gas streams hydrogen sulfide at high concentrations in low empty bed residence times.

### FIELD OF THE INVENTION

The present invention relates to biofilter systems and the biofilter media employed in such systems, as well as, methods of using same to remove odour causing compounds from waste gas streams.

### BACKGROUND OF THE INVENTION

Biofiltration is a known air pollution control technique that has been applied to control odour and remove volatile organic compounds (VOC) from waste gas streams generated by wastewater treatment plants and chemical plants, as well as various rendering, food processing, flavour manufacturing and composting facilities.

In a typical biofilter, a waste gas stream is urged to flow through a moist, biologically active, packed bed. The bed contains microorganisms that are immobilised on a thin biofilm that is formed on the surface of the packing material. The microorganisms serve as the biocatalysts in the contaminant degradation process. They transform the air contaminants into biomass and harmless products through their metabolic activities.

The process underlying the operation of a biofilter is a multi-step process that involves phase transfer, adsorption and biodegradation. As a first step, contaminants must be transferred from the gaseous phase to the liquid phase since they cannot be degraded directly while in the gaseous phase. Once in the liquid phase, the contaminants are adsorbed to the packing material or biofilter media (as it is often referred to). Thereafter, the contaminants are biodegraded within the biofilm. The overall efficiency of the biofiltration process is determined by the relative rates of phase transfer, adsorption and the biological reactions.

Selecting the appropriate packing material or biofilter media is critical to ensure proper functioning of the biofilter. While the biofilter media serves multiples purposes, its most important function tends to be providing contact between the gas-phase contaminants and the active microbial colonies immobilized on the biofilm. In considering the suitability of a material for use as a biofilter media, the following factors are considered to be desirable: the ability to support bacterial growth, large surface area, structural integrity (i.e. resistance to compaction), high porosity, low chemical reactivity, pH buffering capacity, good adsorption properties, sufficient water retention capability and non-biodegradability.

Several different biofilter media have been used in the past. These typically fall in one of two categories: naturally bioactive or inert. However, in certain applications, bioactive and inert packing materials have been combined.

Bioactive packing materials typically include soil, peat, compost, bark and manure. These materials can retain water and generally contain enough nutrients to sustain an initial microbial population. These materials have been used in many applications because they tend to be abundantly available and are generally inexpensive. However, this type of biofilter media has encountered various drawbacks in the field. Biofilters using these materials tend to require large filter beds on account of the low biodegradation rate and the significant bulk density of the media that tends to limit the filter bed height. Additionally, these media tend to degrade over time. They lose their water retaining characteristics and settling of the media due to biomass growth tends to occur. Eventually, biofilters using this type of media may experience a loss of performance due to a significant gas phase pressure drop in the media and channeling of the waste gas through the filter bed.

Inert biofilter media are porous materials (either naturally occurring or synthetic) that usually require inoculation of microorganisms. Examples of inert biofilter media that have been used in previous biofilter applications include activated carbon, gas-aerated concrete, gravel, lava rock, ceramics and polymeric foams. Some synthetic biofilter media have yielded better contaminant removal rates and generally performed better than bioactive packing materials. This is due in part to the fact that they tend to have a larger surface area and have been able to achieve a better distribution of gas flow through the media. However, clogging, compaction and excessive gas-phase pressure drop due to extensive biomass growth still remain a serious problem for these types of biofilter media. These issues can severely impact performance of the biofilter causing a decline in the contaminant removal efficiency.

An example of an engineered (synthetic) packing material is described in European Patent No. 0 497 214 of Fattinger. The biofilter media of Fattinger has a hydrophilic core coated with a hydrophobic layer. The hydrophilic core is populated by microorganisms. It is a granular material made from a porous substance, such as gas-aerated concrete, swelling clay or pumice, whereas the hydrophobic layer can be activated charcoal or adsorption resin. A bonding agent may also be used when applying the hydrophobic layer to the hydrophilic core. Fattinger also discloses that this biofilter media may be used to purify exhaust air containing toluene, xylene, ethyl acetate and benzene. While this packing material tends to have better structural and biological properties than wood-based packing materials, it tends to suffer from the clogging problem described above as well as the acid solubility of gas-aerated concrete. In addition, this packing material tends to have a relatively high density resulting in increased shipping costs associated therewith.

A biofilter system using the packing material of Fattinger to remove hydrogen sulfide from waste gas streams was described in U.S. Pat. No. 6,358,729 of Ferranti. The patent describes a compact plant unit for the deputation of air polluted with odorous substances, such as hydrogen sulfide, mercaptans and dimethyl disulfide. The plant includes a prescrubbing section, a filtering bed and post-scrubbing section, all placed in sequence. The filtering bed of Ferranti preferably consists of particles of a filtering material made in accordance with European Patent No. 0 497 214 of Fattinger. Ferranti describes that very high H₂S removal efficiencies may be achieved using this compact plant unit. While the compact unit plant of Ferranti was found to be effective in removing hydrogen sulfide, its empty bed residence time (EBRT) for the H₂S removal at high concentrations tended to be high.

The engineered biofilter media described in United States Patent Publication No. 2005/0084949 of Shareefdeen et al. and currently made commercially available by the assignee of the present application, BIOREM Technologies Inc. of Guelph, Ontario under the name BIOSORBENS™, has had greater success in removing hydrogen sulfide from waste gas streams. Shareefdeen et al. disclose a biofilter media that has a porous hydrophilic nucleus and a hydrophobic coating on the hydrophilic nucleus. The hydrophilic nucleus is formed of aggregates whose primary ingredients preferably include silica and alumina. The hydrophobic coating includes a metallic agent, microorganisms, nutrients, organic carbon, an alkaline buffer, a bonding agent, an adsorptive agent, and a hydrophobic agent. The inclusion of a metallic agent (preferably iron) in the biofilter media of Shareefdeen et al. allows the removal of sulfur by the formation of iron sulfide and also serves to enhance the conversion and biological processing of sulfur compounds in the contaminated air. The metallic agent acts as catalyst to increase the rate of biological oxidation and enhance the activity of the microorganisms. As a result, the biofilter media of Shareefdeen et al. has shown an improved ability to remove higher concentrations of hydrogen sulfide at lower EBRTs and has achieved a higher H₂S removal efficiency than the packing material of Fattinger.

In light of the foregoing, it would be advantageous if a biofilter system were capable of achieving even higher removal rates of hydrogen sulfide at greater concentrations with lower EBRTs than conventional biofilter systems. Moreover, it would be desirable if the biofilter media used in such a system could be engineered to optimize its physical, material and biological properties for improved performance and versatility.

### SUMMARY OF THE INVENTION

In accordance with a broad aspect of an embodiment of the present invention, there is provided a biofilter media having a plurality of expanded glass granules. Each expanded glass granule has a coating thereon. The coating includes a bonding agent, an adsorptive agent, microorganisms and nutrients. In an additional feature, each expanded glass granule measures between 2 mm and 40 mm and preferably, between 8 mm and 16 mm. In a further feature, the bonding agent is cement. In still another feature, the adsorptive agent is activated carbon.

Additionally, the microorganisms and nutrients are provided by at least one of peat and compost. In yet another feature, the microorganisms are provided by compost and include at least one of Pseudomonas pseudoalcaligenes, Pseudoxanthomonas and Paenibaccilus lautus. In a further feature, the microorganisms are provided from a source of inoculants and include at least one of Thiobacillus thioparus and Thiobacillus thiooxidans.

In an additional feature the nutrients include phosphorus, nitrogen and potassium and may further include zinc acetate.

In another feature, the coating on the expanded glass granule further includes an acid. Optionally, the acid may be phosphoric acid.

In another broad aspect of an embodiment of the present invention, there is provided a method for removing odour causing compounds from a waste gas stream. In accordance with this method, a biofilter system having a biofilter media is provided. The biofilter media includes a plurality of expanded glass granules. Each expanded glass granule has a coating thereon. The coating includes a bonding agent, an adsorptive agent, microorganisms and nutrients. The waste gas stream is urged to flow through the biofilter media of the biofilter system. In an additional feature, the odour causing compounds are selected from the group consisting of: (a) hydrogen sulfide; (b) reduced sulfur compounds; and (c) volatile organic compounds. In another feature, the odour causing compounds are reduced sulfur compounds selected from the group consisting of: (a) methyl mercaptan; (b) dimethyl sulfide; and (c) dimethyl disulfide.

In still another broad aspect of an embodiment of the present invention, there is provided a biofilter system. The biofilter system has a housing and an inlet provided to the housing for receiving contaminated air. An outlet is also provided to the housing for exhausting cleaned air. The biofilter system also includes a biofilter media situated between the inlet and the outlet through which the contaminated air flows. The biofilter media has a plurality of expanded glass granules. Each expanded glass granule has a coating thereon. The coating includes a bonding agent, an adsorptive agent, microorganisms and nutrients.

In an additional feature, the biofilter includes a water delivery system for providing moisture to the biofilter media. The moisture provided by the water delivery system is in the form of one of water and steam. The water delivery system includes a steam generator for supplying steam to the biofilter media and may also include irrigation conduits to deliver the water to the biofilter media. Nozzles are operatively connected to the irrigation conduits for spraying water onto the biofilter media. Also provided is a flow meter for controlling the flow of water through the irrigation conduits.

In another feature, the housing includes a drain line in fluid communication with the biofilter media for removing excess water therefrom.

In still another feature, the biofilter system includes sensor means operatively connected to the biofilter media. The sensor means may include a temperature sensor for measuring the temperature of the biofilter media and a pressure sensor for measuring the pressure at which the contaminated air flows through the biofilter media. Optionally, the sensor means includes a pH monitoring probe. The pH monitoring probe is disposed in the drain line.

In a further feature, the biofilter system includes a control system operatively connected to the water delivery system and the sensor means. The control system is operable to actuate the water delivery system in response to input received from the sensor means. Additionally, the sensor means may include a temperature sensor for measuring the temperature of the biofilter media and a pressure sensor for measuring the pressure at which the contaminated air flows through the biofilter media. The control system may be operable to actuate the water delivery system to adjust the moisture being delivered to the biofilter in response to input received from the temperature sensor or the pressure sensor.

In still another feature, the sensor means includes a pH monitoring probe for measuring the pH of the biofilter media. The control system is operable to adjust the pH of the biofilter media in response to input received from the pH monitoring probe.

In yet another feature, the biofilter system includes a humidification chamber disposed within the housing between the inlet and the biofilter media for moistening the contaminated air prior to entry of the contaminated air into the biofilter media. The contaminated air is moistened within the humidification chamber using one of: (a) a pneumatic spray; (b) high-pressure water; and (c) steam. Also provided is a steam generator operatively connected to the humidification chamber for delivery of steam thereto.

### BRIEF DESCRIPTION OF THE DRAWINGS

The embodiments of the present invention shall be more clearly understood with reference to the following detailed description of the embodiments of the invention taken in conjunction with the accompanying drawings, in which:

FIG. 1 is a simplified illustration of a biofilter system having a biofilter media produced in accordance with an embodiment of the present invention;

FIG. 2 is a conceptual illustration of a granule of the biofilter media shown in accordance with an embodiment of the present invention;

FIG. 3 is a graphical representation of the elimination capacity vs. the loading rate for a biofilter system having the biofilter media provided in accordance with an embodiment of the present invention, treating hydrogen sulfide; and

FIG. 4 is a graphical representation showing the hydrogen sulfide removal efficiency of a biofilter system having the biofilter media provided in accordance with an embodiment of the present invention, plotted against the hydrogen sulfide concentration in the waste gas stream and specified empty bed residence times (EBRT).

### DETAILED DESCRIPTION OF EMBODIMENTS OF THE INVENTION

The description which follows, and the embodiments described therein are provided by way of illustration of an example, or examples of particular embodiments of principles and aspects of the present invention. These examples are provided for the purposes of explanation and not of limitation, of those principles of the invention. In the description that follows, like parts are marked throughout the specification and the drawings with the same respective reference numerals.

In the following specification, the terms “biofilter” or “biofilter system” refer to a system that employs microorganisms to effect biodegradation of contaminants in a waste gas stream. The term “biofilter media” refers to the packing material used in the filter beds of such systems. Furthermore, the term “contaminants” or “air contaminants” refer to chemical compounds present in waste gas streams and includes, but is not limited to, sulfur-based compounds, such as hydrogen sulfide (“H₂S”), organic sulfides, reduced sulfur compounds, for instance, methyl mercaptan, dimethyl sulfide and dimethyl disulfide, and volatile organic compounds (“VOCs”), such as aliphatic and aromatic compounds. Further, the terms “contaminated air stream” or “waste gas stream” refer to a flow of air/gas that contains contaminants.

Biofilter System

FIG. 1 shows a simplified illustration of a biofilter system 20 according to an embodiment of the invention. The biofilter system 20 includes a housing 22 that encloses a biofilter bed 24. The biofilter system 20 may be placed above or below ground and may be operated under positive or negative pressure with or without covers.

The biofilter bed 24 has a base 26 upon which rests a column 28 of biofilter media 30. A waste gas inlet 32 provides access to the housing 22 thereby allowing a contaminated air stream to enter the biofilter bed 24. Positioned adjacent to the waste gas inlet 32 is a humidification chamber 34. The biofilter system 20 further includes an outlet 36 to allow a cleaned air stream to exit the housing 22 following treatment in the biofilter bed 24.

A water supply system 40 is operatively connected to the housing 22 to provide the required moisture (in the form of water and/or steam) to the biofilter media 30. The water supply system 40 includes a water inlet 42 for receiving water to be used for either steam generation or irrigation of the biofilter media 30. The water inlet 42 may supply water to a steam generator 44 through conduit 45. The steam generator 44 is operable to generate steam (when required) and supply it to the humidification chamber 34 via conduit 47 and waste gas inlet 32. Irrigation conduits 46 attached to the water inlet 42 are used to deliver water into the housing 22. The irrigation conduits 46 are furnished with spray nozzles 48 for spraying water on the biofilter media 30.

The water supply system 40 may also include a flow meter 50 disposed downstream of the water inlet 42 to control the amount of water that enters the biofilter system 20 and more specifically, the irrigation conduits 28. A drain line 52 disposed within the housing 22 allows for the removal of excess water and any waste accumulated during the cleansing and irrigation of the biofilter media 30.

In the present embodiment, the biofilter system 20 includes one or more media temperature sensors 56 (only one sensor shown) that measure the temperature of the biofilter media and one or more pressure sensors 58 (only one sensor shown) that measure the pressure at which the waste gas is flowing through the biofilter media 30. A biofilter control system 60 governs the operation of the biofilter system 20 and communicates with the media temperature sensor 56, the pressure sensor 58, and the water supply system 40. As will be explained in greater detail below, the biofilter control system 60 may actuate the water supply system 40 in response to an input signal it receives from the temperature sensor 56. More specifically, the control system 60 may adjust the temperature of the biofilter media 30 by selectively adding steam to the contaminated air stream in the waste gas inlet 32 or in the humidification chamber 34, or by irrigating the biofilter media 30 with water.

The biofilter system 10 may also have a pH monitoring probe (not shown) disposed in the outlet 36 to monitor the operating environment of the biofilter media 14. A more detailed description of the biofilter system 20 can be found in U.S. Pat. No. 5,869,323, the content of which is hereby incorporated herein by reference.

Biofilter Media

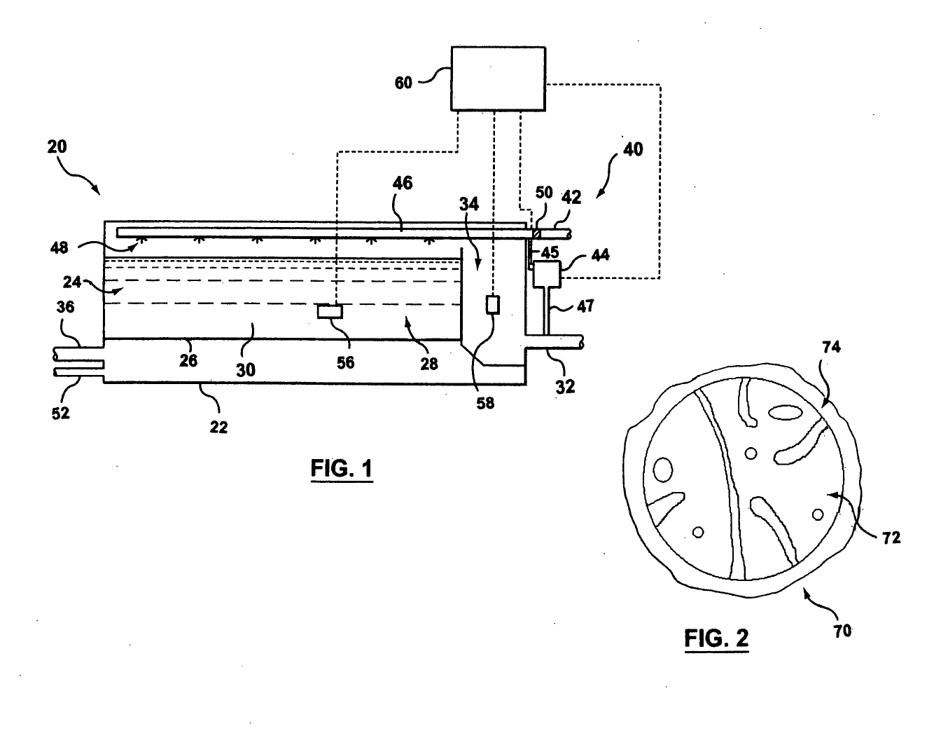

Within the biofilter system 30, the biofilter media 30 is provided to remove contaminants from the contaminated air stream received within the housing 22. FIG. 2 conceptually illustrates a granule, bead or pellet 70 of the biofilter media 30 according to an embodiment of the invention. Each granule 70 provides a surface area upon which may be supported the biofilm containing the microorganisms required to biodegrade the contaminants. Each granule 70 is an expanded glass granule 72 that has a hydrophobic coating 74 thereon.

The expanded glass granule 72 is stable, inorganic non-reactive, non-flammable, non-toxic, non-odorous, non-biodegradable and acid resistant. In addition, the expanded glass granule 72 tends to be relatively hard and rigid which allows it to better resist compaction from biomass growth and avoid high-gas phase pressure drop that may adversely impact on biofilter performance. By virtue of its relatively high porosity, the expanded glass granule 72 tends to exhibit excellent moisture retention properties and has a relatively low bulk density. The expanded glass granule 72 is primarily composed of silica and alkali oxides (i.e. predominantly sodium oxide (Na₂O) and to a lesser extent, potassium oxide (K₂O)) with the remainder being composed of calcium oxide (CaO), alumina (Al₂O₃) and magnesium oxide (MgO). The chemical composition of the expanded glass granule of this embodiment is set out below:

| Compound | % (by weight) |
|---|---|
| Silica (SiO₂) | 71 |
| Sodium Oxide (Na₂O) | 14 |
| Potassium Oxide (K₂O) | 1 |
| Calcium Oxide (CaO) | 9 |
| Alumina (Al₂O₃) | 3 |
| Magnesium Oxide (MgO) | 2 |

In this embodiment, the expanded glass granule 72 is a manufactured and shaped granule having a generally spherical shape. The expanded glass granule 72 may be sized between 2 mm and 40 mm. However, preferably it measures between 8 mm and 16 mm. The expanded glass granulate product made commercially available by Dennert Poraver GmbH of Schlüsselfeld, Germany under the name PORAVER™ has been found to be suitable for use as the expanded glass granule 72. This product is manufactured from recycled glass and has been used in the past as a component of building materials such as plasters, mortars, adhesives and fillers. However, it will be appreciated that other granulate products exhibiting similar material properties and having different chemical compositions could also be employed to advantage.

The coating 74 includes a bonding agent for bonding the coating to the expanded glass granule 72, an adsorptive agent, microorganisms and nutrients. In the preferred embodiment, the bonding agent is an alkaline bonding agent such as, cement or other like cementitious material. Preferably, the bonding agent will have the following composition: tricalcium silicate (˜50%), dicalcium silicate (25%), tricalcium aluminate (10%), tetracalcium aluminoferrite (10%), and gypsum (5%). It will however be understood that the composition of the bonding agent may be adjusted to accommodate the chemical make-up of a particular waste gas stream. For instance, in the case of higher sulfur loadings, a bonding agent with a lower level of tricalcium aluminate may be employed.

The adsorptive agent may be one or more of activated carbon (a form of inorganic carbon), adsorption resin and clinoptilolite (natural or synthetic). Preferably, some quantity of activated carbon is used because it increases the adsorption of chemicals such as reduced sulfides and aliphatic and aromatic compounds. The use of clinoptilolite may also be desirable due to its capacity for elevated cation exchange which tends to make it adaptable to different field applications. In addition, clinoptilolite is provided with a large surface area and can adsorb gases including hydrogen sulfide, ammonia, mercaptans, formaldehyde, and VOC gases from contaminated air streams.

The microorganisms present in the coating may be aerobic mesophilic bacteria or thermophilic bacteria. In applications where mesophilic bacteria populate the biofilter media 30, the biofilter system 20 may be operated at temperatures in the range of 20° C. to 40° C. Where the coating 74 includes thermophilic bacteria, an operating temperature of greater than 45° C. may be maintained in the biofilter bed 24. This can be achieved by supplying steam to biofilter media 30, for example.

The microorganisms can be supplied to the biofilter media 30 in various ways. An organic substrate such as peat or compost (which contain microorganisms) and nutrient solution may be added to the mixture of expanded glass granule 72 and coating 74 during manufacturing of the biofilter media 30. In applications where compost is added to the mixture, it may not be necessary to inoculate the biofilter media 30 since contaminants can be biodegraded using the natural microbial populations present in the compost. Such bacteria may include Pseudomonas pseudoalcaligenes, Pseudoxanthomonas and Paenibacillus lautus. These microorganisms tend to be effective in breaking down different sulfur compounds present in the waste gas and have been shown to achieve efficient contaminant removal without requiring the addition of further microorganisms by inoculation. It will however be appreciated that in certain applications it may be advantageous to supplement the naturally-occurring bacteria with additional microorganisms through inoculation of the biofilter media 30. This may be carried out to generally improve the performance of the biofilter system or to specifically enhance degradation of a particular compound or group of compounds.

In other applications, the microorganisms may be provided by a single strain or mixed culture of inocula grown in a separate bioreactor. The source of inoculants may be a standard laboratory bacterial growth medium such as agar or broth. These microorganisms could be added to the coating 74 in liquid form either during manufacturing of the biofilter media 30 or during the operation of the biofilter system 20 (via the water delivery system 40). For instance, the biofilter media 30 may be inoculated with the following bacteria: Thiobacillus thioparus, begigiatoa, thiothrix genera, and T. feroxidants.

As will be appreciated by persons skilled in the art, a wide variety of nutrients for the microbial culture may be used. Such nutrients may include a source of organic carbon and a blend of nitrogen, phosphorus and potassium compounds, as well organic and inorganic compounds and other ingredients that may tend to support and promote bacterial growth and encourage degradation of certain contaminants. Examples of such ingredients include magnesium, manganese, inorganic or organic sulfur, calcium, iron, copper, cobalt, zinc, boron and molybdenum. In particular, the addition of zinc acetate to the biofilter media 30 during manufacture has been found to improve the removal of reduced sulfur compounds, in particular, dimethyl sulfide, from the contaminated air stream. By providing an appropriate balance of nutrients and by adjustment of nutrient concentration, it is possible to achieve high levels of growth of bacteria and thus accelerated rates of contaminant degradation.

Other additives may also be included to the coating 74, for example, to adjust the pH of the biofilter media 30 to the desired value. In certain applications, it may be advantageous to add an acid during the coating process. The acid may be an organic acid or an inorganic acid. However, preferably, the acid employed is phosphoric acid (H₃PO₄). It has been found that phosphoric acid tends to increase the porosity and the buffering properties of the biofilter media 30. Moreover, the addition of phosphoric acid also tends to significantly increase the surface area and adsorption capacity of the biofilter media 30, allowing for better retention and bonding of the air contaminants. As an added benefit, the phosphorus from the phosphoric acid may also serve as a nutrient source to support microorganism growth. In other applications where maintaining a neutral pH is desired, a neutralizing alkaline agent may be added to the biofilter media during the manufacture of same.

Advantageously, the expanded glass granule 72 with its coating 74 has a relatively lower weight and lower density than the coated hydrophilic nucleus of known biofilter media. For instance, whereas the bulk density of the coated expanded glass granule 72 is 0.29 kg/L on a dry weight basis, the bulk density for the coated hydrophilic nucleus of the biofilter media described in United States Patent Application Publication No. 2005/0084949 and currently made commercially available by the assignee of the present application, BIOREM Technologies Inc. of Guelph, Ontario under the name BIOSORBENS™, is 0.65 kg/L on a dry weight basis. It will thus be appreciated that the biofilter media 30 of the present embodiment is approximately 56% lighter than the BIOSORBENS™ biofilter media. The relatively light-weight/low density characteristics of the biofilter media 30 tend to facilitate handling of the biofilter media when charging and discharging the biofilter media 30 in the biofilter bed 24 and during maintenance and servicing operations. In particular, the biofilter media 30 may be removed from the biofilter bed 24 to permit the excess biomass collected on the surface of the biofilter media to be washed off thereby allowing recycling of the biofilter media. In this way, the clogging problems typically associated with conventional biofilter packing materials tend to be mitigated in the biofilter media 30.

In addition, freight costs associated with the biofilter media tend to be lower than those associated with the heavier conventional biofilter media thereby enhancing the cost effectiveness of the biofilter media 30.

Operation

The operation of the biofilter system 20 will now be described in greater detail. The biofilter system 20 is supplied with a waste gas stream from, for example, a rendering plant. The contaminated air enters the housing 22 through the waste gas inlet 24 typically under pressure, either positive or negative, (preferably, approximately −12 to 12 inches of water column), such that it is urged to flow through the biofilter bed 26.

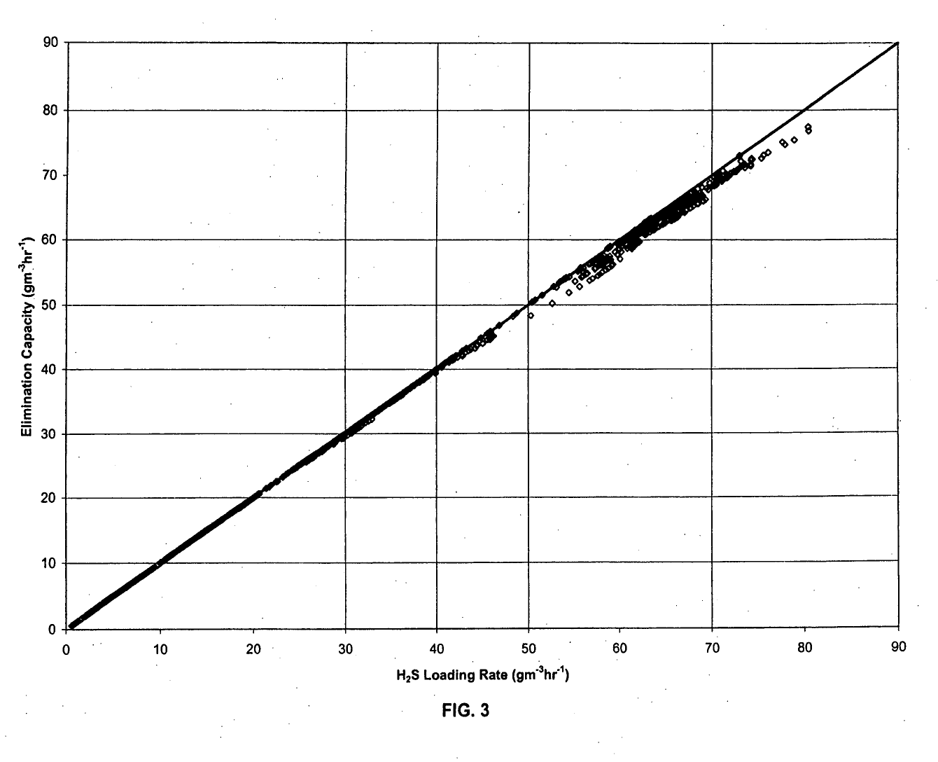

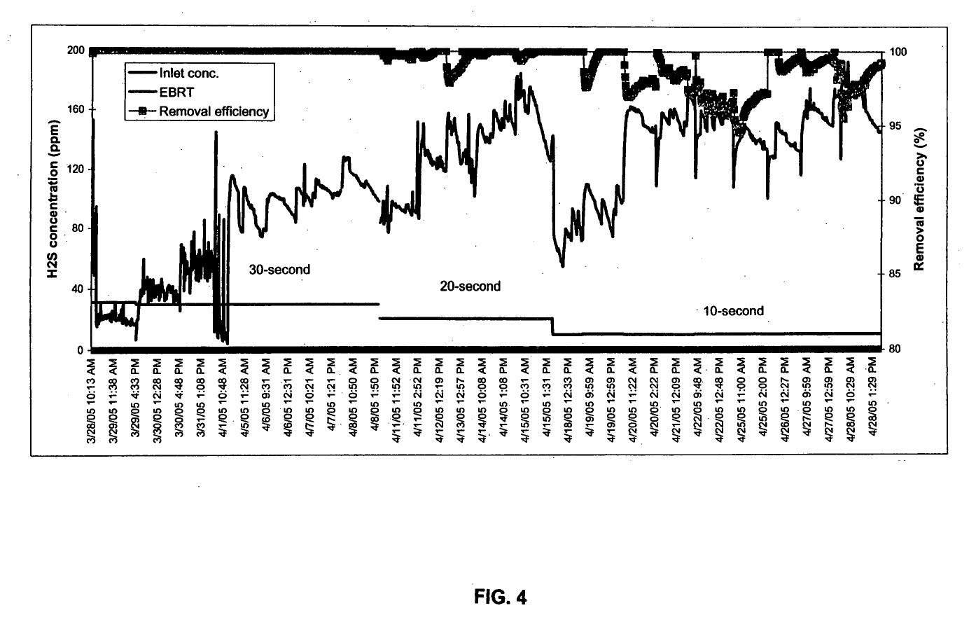

As the waste gas stream flows through the biofilter media 30, contaminants undergo phase transfer from the gas phase to the liquid phase. In the biofilter system of the present embodiment, the phase transfer of hydrogen sulfide tends to occur more rapidly in the biofilter media 30 than in conventional biofilter media. It is believed that the higher rate of phase transfer of hydrogen sulfide is due to its particular affinity for the coated expanded glass granule 72. This increased affinity for the coated expanded glass granule 72 allows the biofilter system 20 to achieve higher removal efficiencies (elimination capacities) for hydrogen sulfide than were previously obtained with biofilter systems employing conventional biofilter media. An example of the elimination capacity for hydrogen sulfide at various concentrations is shown in FIG. 3.

Once the contaminants have transitioned to the liquid phase, the contaminants are adsorbed onto the biofilm formed on the surface of the expanded glass granule 72 and then degraded by the metabolic activities of the microorganisms. Carbon dioxide and water are produced as a result of the biological oxidation of VOCs. The sulfur-based compounds may break down into sulfites (SO₃²⁻), sulfates (SO₄²⁻), sulfides (S²⁻) or sulfur (S). The water soluble sulfur compounds can be easily flushed out of the biofilter bed 24 without the use of chemicals by washing out the biofilter media 30 with water, using irrigation at intermittent intervals.

The coarse granular configuration of the expanded glass granule 72 as well as its characteristic low density/light weight tends to permit easy washing of the biofilter media to remove not only the products of the contaminant degradation but also any excessive biomass which may have accumulated on the surface of the biofilter media 30. The problems associated with high gas flow resistance and clogging encountered in known biofilter media tend to be minimized in the biofilter media 30. Accordingly, the biofilter media may be recycled, regenerated and reused with relative ease thus tending to impart to it a relatively long service life.

Advantageously, the residue water from the periodic flushing of the biofilter media 30 can be discharged from the biofilter bed 24 through the drain line 52.

In this embodiment, the water content in the biofilter media 30 may be adjusted by humidifying the air stream prior to its entry into the biofilter bed 24 and/or irrigating the surface of the biofilter media 30. Humidification of the air stream may occur in the humidification chamber 34 using, for example, one of the following moisture delivery systems: a pneumatic spray, high-pressure water or steam (not shown). The delivery of moisture to the biofilter media 30 may be accomplished through the water supply system 40, more specifically, via the irrigation conduits 46 and the spray nozzles 48.

During operation of the biofilter system 20, the temperature sensor 56 detects the temperature of the biofilter media 30 and transmits a signal to the control system 60 which may actuate the moisture delivery system or the water supply system 40 in response to that signal to cause water and/or steam to be delivered to the air stream or directly to the biofilter media. In this way, the temperature of the biofilter media may be maintained in the optimal range to best promote the sustained growth and development of the microorganisms.

The delivery of moisture in the form of water or steam may be actuated by the control system 60 in response to a signal received from the pressure sensor 58. For instance, if the pressure sensor 58 detects pressure at a particular point across the biofilter media 30 which exceeds the desired pressure range, this may be an indication that sulfur has accumulated excessively on the surface of the biofilter media 30 in that area thereby impeding proper gas flow through the media. In this case, the biofilter control system 60 may cause the water supply system 40 to irrigate the biofilter media 14 with water to wash away the sulfur build-up.

The control system 40 may also be configured to monitor other parameters in the biofilter media 30 to ensure the optimal operating conditions are maintained within the biofilter bed 24. For instance, the biofilter system 20 can include a pH monitoring probe (not shown) to periodically measure the pH in the biofilter bed 24. If the pH value measured falls outside of the desired range, an appropriate chemical solution, such as a liquid buffer, may be added through the water supply system 40. Other sensors could also monitor the need for further nutrients—these could be delivered through the water supply system 40.

Unlike conventional biofilter systems, the biofilter system 20 tends to have a shortened or reduced acclimation period. The biofilter system 20 can begin removing H₂S within less than a day and become fully operational within 48 hours of the start up operation. This reduced acclimation period is due to the fact that the expanded glass granule 72 tends to exhibit improved water retention properties and tends to present a larger and rougher surface area than known biofilter media thereby tending to enhance adhesion of the microorganisms onto the biofilter media 30. The increased surface area and roughness in combination with the improved water retention properties of the expanded glass granule tends to favour more rapid initial microbial colonization resulting in shortened biofilter start-up time. As a result of the reduced start-up time, it is possible to use a smaller volume of biofilter media to treat a given volume of contaminated air.

With its light weight/low density characteristics, the biofilter media 30 allows for greater flexibility in the design of biofilter systems. More specifically, the biofilter media 30 can be used to lighten the overall weight of a biofilter system thereby lessening the need for more structural support (i.e. larger and heavier foundations). In turn, this makes it possible to install such biofilter systems in a variety of locations, including on roof tops. In addition, in biofilter systems that employ the biofilter media 30, the height of the column in the biofilter bed may be increased to permit greater bed depth. This may allow the installation footprint of the biofilter system to be reduced for even greater versatility. Additionally, increased bed height may be advantageous in the case where one or more compounds are not degraded until after other compounds have been broken down to very low concentrations. In such cases, spatial separation of zones for the biodegradation of different compounds as a function of height in a biofilter bed tends to result.

The removal kinetics of various contaminants using a biofilter system having the biofilter media 30 in accordance with an embodiment of the present invention have been examined through performance data obtained in laboratory during initial pilot studies The performance of the biofilter media 30 was compared to that of BIOSORBENS™, a known, high performance biofilter media currently made commercially available by the assignee of the present application, BIOREM Technologies Inc. of Guelph, Ontario.

The findings obtained from the different studies are described as follows:

Using a biofilter system constructed and operated in accordance with the principles of the present invention, high H₂S removal efficiency at high inlet concentrations in low empty bed residence times (EBRT) has been consistently obtained. More specifically, the biofilter system has achieved greater than 95% removal of 200 ppm of H₂S in 10 to 30 seconds EBRT. In a 30 seconds EBRT, the biofilter system successfully removed greater than 99% of 200 ppm of H₂S. The presence of reduced sulfur compounds in the waste gas stream did not appear to affect the removal efficiency of the hydrogen sulfide. In comparison, the high performance biofilter media currently made commercially available by the assignee of the present application, BIOREM Technologies Inc. of Guelph, Ontario under the name BIOSORBENS™, is capable of removing only up to 90% of 150-200 ppm of H₂S in a 30 seconds EBRT.

Performance data for the removal of H₂S at concentrations of up to 100 ppm with the biofilter media provided in accordance with the principles of the present embodiment (identified as “LWE”) and with the known BIOSORBENS™ media are compared in Table 1 below:

TABLE 1
Removal Efficiencies of H₂S using the biofilter media provided in
accordance with the principles of the present invention and the known
BIOSORBENS ™ biofilter media

| H₂S | Removal efficiencies (%) | | | |
|---|---|---|---|---|
| concentration | 30-second EBRT | | 20-second EBRT | |
| (ppm) | LWE | BIOSORBENS ™ | LWE | BIOSORBENS ™ |
| 10 | 100 | 100 | 100 | 100 |
| 20 | 100 | 100 | 100 | 96 |
| 30 | 100 | 100 | 100 | 88 |
| 40 | 100 | 98 | 100 | 81 |
| 50 | 100 | 94 | 100 | 76 |
| 60 | 100 | 91 | 100 | 71 |
| 70 | 100 | 87 | 100 | 68 |
| 80 | 100 | 84 | 100 | 64 |
| 90 | 100 | 81 | 100 | 61 |
| 100 | 100 | 79 | 100 | 59 |

### As will be appreciated, the biofilter media provided in accordance with the principles of the present invention exhibits improved removal efficiencies.

In addition, it has been shown that the biofilter media provided in accordance with the principles of the present invention is capable of handling peak concentrations of H₂S of up to 400 ppm while the known BIOSORBENS™ is effective up to peak concentrations of about 100 ppm.

It was found that the removal of dimethyl sulfide could be significantly improved by adding a predetermined quantity of zinc acetate to the biofilter media during the manufacture thereof. More specifically, with the addition of zinc acetate, it was possible to increase the rate of removal of dimethyl sulfide to from 25% to 55% at 30 seconds EBRT.

It will thus be appreciated that the physical, material and biological characteristics of the biofilter media 30 as described above enable the biofilter media to perform better than other, known biofilter media. Whereas some conventional biofilter media are able to achieve satisfactory removal rates for hydrogen sulfide by improving biodegradation of the contaminants, the biofilter media 30 is designed to encourage both phase transfer and enhance biodegradation of the contaminants. As a result, the biofilter media is able to remove hydrogen sulfide and reduced sulfur compounds from waste gas streams with superior efficiency.

Although the foregoing description and accompanying drawings relate to specific preferred embodiments of the present invention as presently contemplated by the inventor(s), it will be understood that various changes, modifications and adaptations, may be made without departing from the spirit of the invention.

What is claimed is:

1. A biofilter media comprising a plurality of expanded glass granules, each expanded glass granule having a coating thereon, the coating including a bonding agent, an adsorptive agent, microorganisms and nutrients.

2. The biofilter media of claim 1 wherein each expanded glass granule measures between 8 mm and 16 mm.

3. The biofilter media of claim 1 wherein the bonding agent is cement.

4. The biofilter media of claim 1 wherein the adsorptive agent is activated carbon.

5. The biofilter media of claim 1 wherein the microorganisms nutrients are provided by at least one of peat and compost.

6. The biofilter media of claim 1 wherein the microorganisms are provided by compost and include at least one of Pseudomonas pseudoalcaligenes, Pseudoxanthomonas and Paenibaccilus lautus.

7. The biofilter media of claim 1 wherein the microorganisms are provided from a source of inoculants.

8. The biofilter media of claim 7 wherein the microorganisms include at least one of Thiobacillus thioparus and Thiobacillus thiooxidans.

9. The biofilter media of claim 1 wherein the nutrients include phosphorus, nitrogen and potassium.

10. The biofilter media of claim 1 wherein the nutrients include zinc acetate.

11. The biofilter media of claim 1 wherein the coating further includes an acid.

12. The biofilter media of claim 11 wherein the acid is phosphoric acid.

13. A method for removing odour causing compounds from a waste gas stream, the method comprising:
providing a biofilter system having a biofilter media, the biofilter media including a plurality of expanded glass granules, each expanded glass granule having a coating thereon, the coating including a bonding agent, an adsorptive agent, microorganisms and nutrients; and
urging the waste gas stream to flow through the biofilter media of the biofilter system.

14. The method of claim 13 wherein each expanded glass granule measures between 8 mm and 16 mm.

15. The method of claim 13 wherein the bonding agent is cement.

16. The method of claim 13 wherein the adsorptive agent is activated carbon.

17. The method of claim 13 wherein the microorganisms are provided by at least one of peat and compost.

18. The method of claim 13 wherein the microorganisms are provided by compost and include at least one of Pseudomonas pseudoalcaligenes, Pseudoxanthomonas and Paenibaccilus lautus.

19. The method of claim 13 wherein the microorganisms are provided from a source of inoculants.

20. The method of claim 19 wherein the microorganisms include at least one of Thiobacillus (T) thioparus and Thiobacillus thiooxidans.

21. The method of claim 13 wherein the nutrients include phosphorus, nitrogen and potassium.

22. The method of claim 13 wherein the nutrients include zinc acetate.

23. The method of claim 13 wherein the odour causing compounds are selected from the group consisting of: (a) hydrogen sulfide; (b) reduced sulfur compounds; and (c) volatile organic compounds.

24. The method of claim 13 wherein the odour causing compounds are reduced sulfur compounds selected from the group consisting of: (a) methyl mercaptan; (b) dimethyl sulfide; and (c) dimethyl disulfide.

25. A biofilter system comprising:
a housing;
an inlet provided to the housing for receiving contaminated air;
an outlet provided to the housing for exhausting cleaned air; and
a biofilter media situated between the inlet and the outlet through which the contaminated air flows, the biofilter media having a plurality of expanded glass granules, each expanded glass granule having a coating thereon, the coating including a bonding agent, an adsorptive agent, microorganisms and nutrients.

26. The biofilter system of claim 25 further including a water delivery system for providing moisture to the biofilter media.

27. The biofilter system of claim 25 wherein the moisture provided by the water delivery system is in the form of one of water and steam.

28. The biofilter system of claim 25 wherein the water delivery system includes a steam generator for supplying steam to the biofilter media.

29. The biofilter system of claim 25 wherein the water delivery system includes irrigation conduits to deliver the water to the biofilter media.

30. The biofilter system of claim 29 wherein the water delivery system further includes nozzles operatively connected to the irrigation conduits for spraying water onto the biofilter media.

31. The biofilter system of claim 29 wherein the water delivery system includes a flow meter for controlling the flow of water through the irrigation conduits.

32. The biofilter system of claim 25 wherein the housing includes a drain line in fluid communication with the biofilter media for removing excess water therefrom.

33. The biofilter system of claim 25 further including sensor means operatively connected to the biofilter media.

34. The biofilter system of claim 33 wherein the sensor means includes a temperature sensor for measuring the temperature of the biofilter media.

35. The biofilter system of claim 33 wherein the sensor means includes a pressure sensor for measuring the pressure at which the contaminated air flows through the biofilter media.

36. The biofilter system of claim 33 wherein:
the housing includes a drain line in fluid communication with the biofilter media for removing excess water therefrom; and
the sensor means includes a pH monitoring probe, the pH monitoring probe being disposed in the drain line.

37. The biofilter system of claim 33 further including a control system operatively connected to the water delivery system and the sensor means, the control system being operable to actuate the water delivery system in response to input received from the sensor means.

38. The biofilter system of claim 37 wherein:
the sensor means includes a temperature sensor for measuring the temperature of the biofilter media; and
the control system is operable to actuate the water delivery system to adjust the moisture being delivered to the biofilter in response to input received from the temperature sensor.

39. The biofilter system of claim 37 wherein:
the sensor means includes a pressure sensor for measuring the pressure at which the contaminated air flows through the biofilter media; and
the control system is operable to actuate the water delivery system to adjust the moisture being delivered to the biofilter in response to input received from the pressure sensor.

40. The biofilter system of claim 37 wherein:
the sensor means includes a pH monitoring probe for measuring the pH of the biofilter media; and
the control system is operable to adjust the pH of the biofilter media in response to input received from the pH monitoring probe.

41. The biofilter system of claim 37 further including a humidification chamber disposed within the housing between the inlet and the biofilter media for moistening the contaminated air prior to entry of the contaminated air into the biofilter media.

42. The biofilter system of claim 41 wherein the contaminated air is moistened within the humidification chamber using one of: (a) a pneumatic spray; (b) high-pressure water; and (c) steam.

43. The biofilter system of claim 41 further including a steam generator operatively connected to the humidification chamber for delivery of steam thereto.

44. The biofilter system of claim 25 wherein each expanded glass granule measures between 8 mm and 16 mm.

45. The biofilter system of claim 25 wherein the bonding agent is cement.

46. The biofilter system of claim 25 wherein the adsorptive agent is activated carbon.

47. The biofilter system of claim 25 wherein the microorganisms and nutrients are provided by at least one of peat and compost.

48. The biofilter system of claim 25 wherein the microorganisms include at least one of Pseudomonas pseudoalcaligenes, Pseudoxanthomonas and Paenibaccilus lautus.

49. The biofilter system of claim 25 wherein the microorganisms are provided from a source of inoculants.

50. The biofilter system of claim 25 wherein the microorganisms include at least one of Thiobacillus thioparus and Thiobacillus thiooxidans.

51. The biofilter system of claim 25 wherein the nutrients include phosphorus, nitrogen and potassium.

52. The biofilter system of claim 25 wherein the nutrients include zinc acetate.
